(19) United States
(12) Patent Application Publication
(10) Pub. No.: US 2026/0308506 A1
(43) Pub. Date: Oct. 8, 2026

(54) MULTI-CHAMBERED ELECTROPORATION CARTRIDGE AND METHODS OF USE

(71) Applicant: LONZA COLOGNE GMBH, Cologne (DE)

(72) Inventors: Ludger Altrogge, Cologne (DE); Timo Gleissner, Cologne (DE); Frank Morrissey-Wettey, Cologne (DE); Claudiu Todor, Cologne (DE); Martin Stainer, Cologne (DE); Paul Warok, Cologne (DE)

(21) Appl. No.: 19/481,581

(22) PCT Filed: Jun. 18, 2024
(86) PCT No.: PCT/US2024/034474

§ 371 Date: Nov. 5, 2025

Related U.S. Application Data
(60) Provisional application No. 63/509,319, filed on Jun. 21, 2023.

(51) Int. Cl. C12M 1/42; C12N 13/00; C12N 15/87

(52) U.S. Cl. CPC C12M 35/02; C12N 13/00; C12N 15/87

(57) ABSTRACT

The present disclosure provides systems, devices, and methods for electroporating at least one cell in a plurality of cells. A cartridge divided into a plurality of separate chambers is provided, where each chamber of the plurality of separate chambers is configured to hold a media comprising the at least one cell. Each chamber of the plurality of separate chambers is fluidly connected via a fluid exchange path, and are functionally separated by constrictions disposed within the fluid exchange path. Each chamber of the plurality of separate chambers includes electrodes for applying a pulsed electrical field to the at least one cell to achieve electroporation and transfection of the cell with nucleic acids. The constrictions disposed between each chamber prevents electric field interference between adjacent chambers during the electroporation procedures.

### FIELD OF THE INVENTION

The present disclosure provides systems, devices, and methods for electroporating a cell. More particularly, a multi-chambered electroporation cartridge is provided.

### BACKGROUND

Electroporation procedures cause cell death to various degrees depending on the cell type, cargo, and electroporation conditions such as buffer system and pulse parameters. The pulse parameters are adjusted for each cell type and cargo, but cell death often occurs as an unwanted side effect, thus reducing the yield of the desired transfected population.

Transfection of large volumes of cells is often approached with a flow-through strategy that consists of repeated transfection of small, fixed volumes of cells until the entire volume is processed. The volumes processed in each step need to be as large as possible in order to process the entire volume quickly enough, yet also small enough to allow for the required performance. This results in limitations in transfection performance and processing time/efficiency. Furthermore, electroporation and/or transfection efficiencies are often reduced when scaling up from small volumes (i.e., 10-100 microliters) to large volumes (i.e., 0.5-20 milliliters).

What is needed is a system, device, and method for electroporation that increases cell viability, transfection performance, and processing efficiency. The present invention fulfills these needs.

### SUMMARY OF THE INVENTION

In a first embodiment, the present invention provides a device for electroporating at least one cell in a plurality of cells. The device comprises a cartridge divided into a plurality of separate chambers. At least one fluid exchange path fluidly connects adjacent chambers of the plurality of separate chambers. A constriction is disposed within the at least one fluid exchange path for functionally separating adjacent chambers. At least one high voltage electrode is disposed in each chamber of the plurality of separate chambers, and adjacent to the constriction of the at least one exchange path. At least one counter electrode is disposed opposite the high voltage electrode and adjacent to the constriction of the at least one exchange path. The device also includes an inlet.

In a second embodiment, the present invention provides a method of electroporating at least one cell in a plurality of cells. The steps of the method may include: injecting a media containing the at least one cell through an inlet of a cartridge, wherein the cartridge is divided into a plurality of separate chambers; flowing the media through the plurality of separate chambers of the cartridge, including flowing the media through at least one exchange path fluidly connecting adjacent chambers of the plurality of separate chambers; constricting the media as it flows through the at least one exchange path via a constriction disposed within the at least one exchange path, the constriction functionally separating the adjacent chambers; applying an electrical field to the at least one cell via at least one high voltage electrode disposed in each chamber of the plurality of separate chambers and adjacent to the constriction of the at least one exchange path, and via at least one counter electrode disposed adjacent to the constriction of the at least one exchange path and opposite the at least one high voltage electrode; and electroporating the at least one cell.

In a third embodiment, the present invention provides a method of transfecting at least one cell in a plurality of cells. The steps of the method may include: injecting a media containing the at least one cell and at least one nucleic acid through an inlet of a cartridge, wherein the cartridge is divided into a plurality of separate chambers; flowing the media through the plurality of separate chambers of the cartridge, including flowing the media through at least one exchange path fluidly connecting adjacent chambers of the plurality of separate chambers; constricting the media as it flows through the at least one exchange path via a constriction disposed within the at least one exchange path, the constriction functionally separating the adjacent chambers; applying an electrical field to the at least one cell via at least one high voltage electrode disposed in each chamber of the plurality of separate chambers and adjacent to the constriction of the at least one exchange path, and at least one counter electrode disposed adjacent to the constriction of the at least one exchange path and opposite the high voltage electrode; and transfecting the at least one nucleic acid into the at least one cell.

In a fourth embodiment, the present invention provides a system for electroporating at least one cell in a plurality of cells. The system comprises a cartridge divided into a plurality of separate chambers, where each chamber of the plurality of separate chambers are configured to hold at least one cell and are fluidly connected via at least one exchange path. The at least one exchange path has a constriction disposed therein. At least one high voltage electrode is disposed in each chamber of the plurality of separate chambers and adjacent to the constriction of the at least one exchange path. At least one counter electrode is disposed opposite the high voltage electrode and adjacent to the constriction of the at least one exchange path. An inlet is disposed at an end of the at least one exchange path. The inlet includes an injection inlet, a dead volume area, an electrically active area, and at least one inlet electrode pair consisting of a high voltage electrode and a counter electrode. An electroporation device is configured to receive the cartridge and generate an electric pulse via the high voltage electrode and the counter electrode.

### BRIEF DESCRIPTION OF THE DRAWINGS

The following drawings form part of the present specification and are included to further demonstrate exemplary embodiments of certain aspects of the present invention.

FIG. 1A shows a side elevational view of an exemplary embodiment of a multi-chambered electroporation cartridge of the present invention.

FIG. 1B shows exemplary dimensions of the electroporation cartridge of FIG. 1A.

FIG. 1C shows a side cross-sectional view of a fluid exchange path, chambers, and constrictions of the electroporation cartridge of FIG. 1A.

FIG. 1D shows a perspective cross-sectional view of the fluid exchange path, chambers, and constrictions of FIG. 1C.

FIG. 2A shows a side elevational view of another embodiment of a multi-chambered electroporation cartridge of the present invention.

FIG. 2B shows exemplary dimensions of the electroporation cartridge of FIG. 2A, and the arrangement of electrodes within half a body layer of the electroporation cartridge of FIG. 2A.

FIG. 2C shows a side cross-sectional view of a fluid exchange path, chambers, and constrictions of the electroporation cartridge of FIG. 2A.

FIG. 2D shows a diagrammatic view of the chambers and constrictions of FIG. 2C, and the electrical field generated by the electrodes disposed within the chambers.

FIG. 2E shows another diagrammatic view of the chambers, constrictions, and electrodes of FIG. 2D, and the restrictions of the electrical field between chambers caused by the constrictions.

FIG. 3A shows a side cross-sectional view of an inlet of a multi-chambered electroporation cartridge of the present invention.

FIG. 3B shows another side cross-sectional view of the inlet of FIG. 3A.

FIG. 4A shows an overmolded circuit board of a multi-chambered electroporation cartridge of the present invention.

FIG. 4B shows a side cross-sectional view of the overmolded circuit board of FIG. 4A.

FIG. 5 shows a circuit board of a multi-chambered electroporation cartridge of the present invention.

FIG. 6 shows an exemplary method of electroporating and or transfecting a cell using the device of the present invention.

### DETAILED DESCRIPTION OF THE INVENTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

As used herein, “between” is a range inclusive of the ends of the range. For example, a number between x and y explicitly includes the numbers x and y and any numbers that fall within x and y.

Electroporation may include the application of controlled electric pulses (e.g., direct current electrical pulses) to at least one cell in a plurality of cells over a predetermined duration of time. These pulses may be introduced via electrodes to the at least one cell in a plurality of cells in different wave forms, such as a square wave pulse or an exponential decay wave pulse, for example. In applying these controlled electric pulses, a transmembrane potential is induced, which causes the reversible breakdown of the cellular membrane. Transmembrane potential may be described as a difference in electrical potential across a cell's plasma membrane, i.e. between the interior and exterior of a biological cell. The breakdown of the cellular membrane results in permeation, or pore formation, of the cellular membrane. This pore formation allows for the introduction of molecules (i.e. molecules such as dye, or nucleic acids, or biologically active molecules, including oligonucleotides, or peptides) into the cell, also known as transfection. Molecules may continue to be introduced into the cell until the pores close by ending the electroporation procedure (or some time thereafter, if the pores remain in the cells for a period). This process may take place anywhere between a few milliseconds to a few minutes.

During electroporation, the external molecules are introduced into the cells from an aqueous solution, which may be a buffer solution specifically adapted to the cells, or a cell culture medium. The temporary pores formed in the cell membrane from the pulse applications allow the biologically active molecules to first reach the cytoplasm in which they may already perform their function or exert any therapeutic action to be examined, and to then also reach the cell nucleus, as is required in certain applications, such as gene therapy applications. The electroporation and/or transfection procedures take place within a cartridge containing electrical field-generating components for facilitating such electroporation and/or transfection procedures.

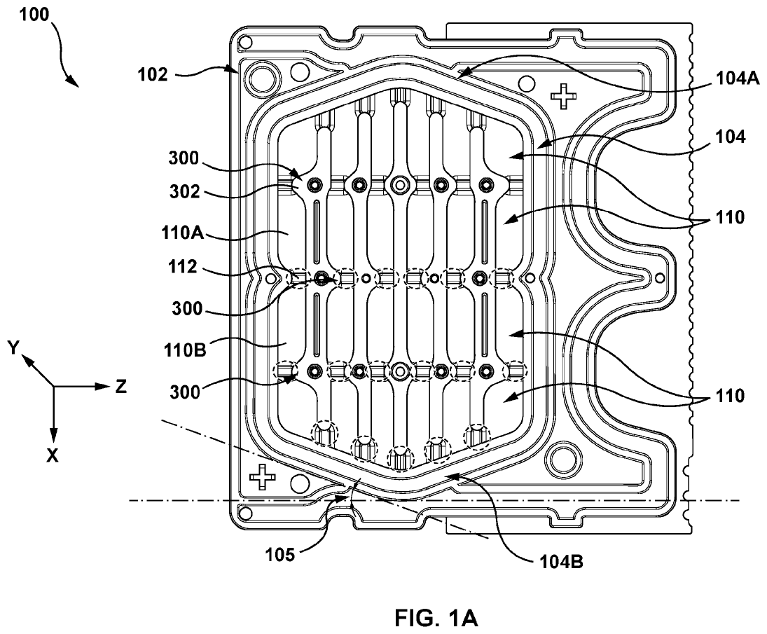

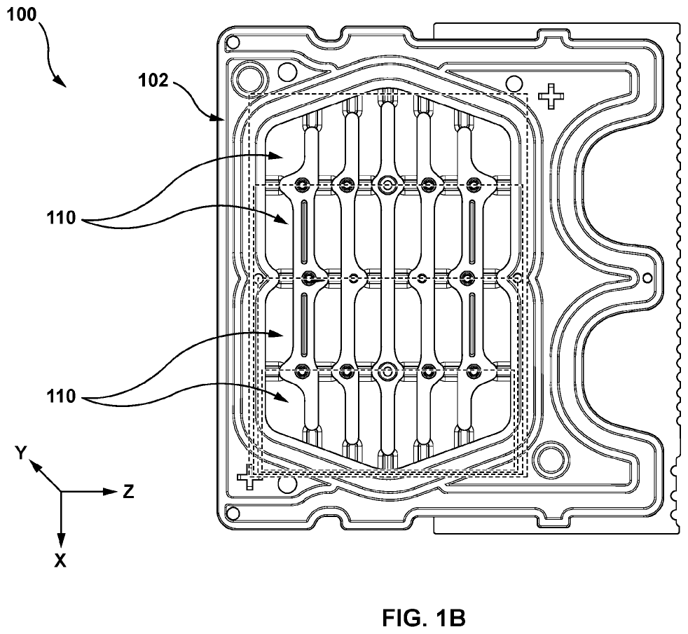

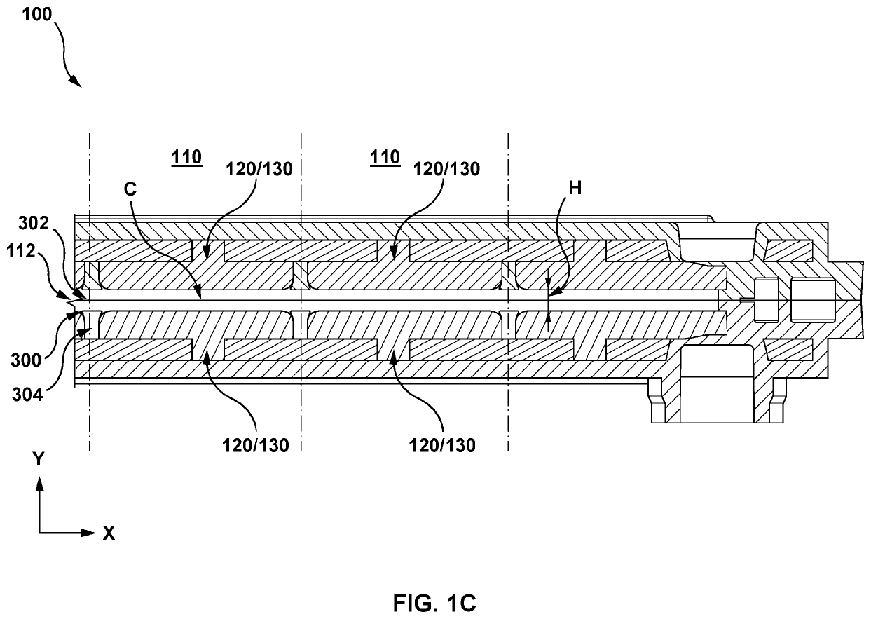

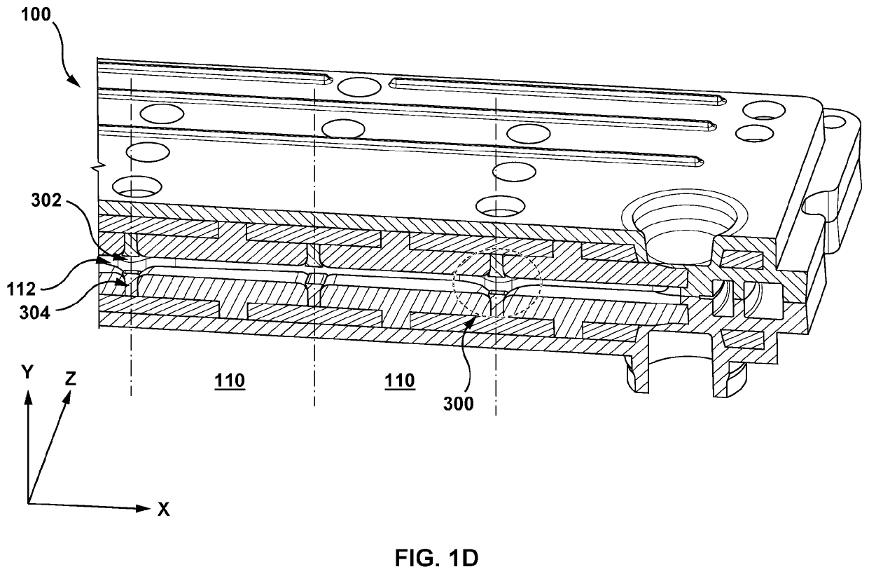

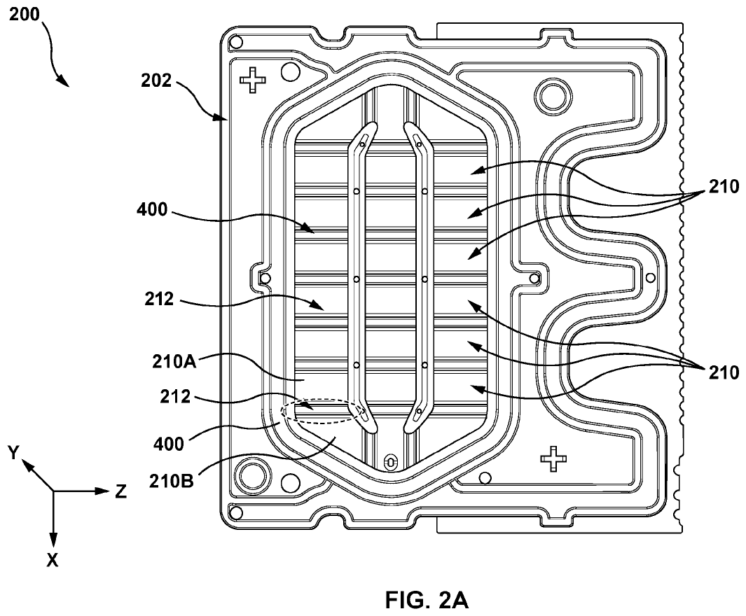

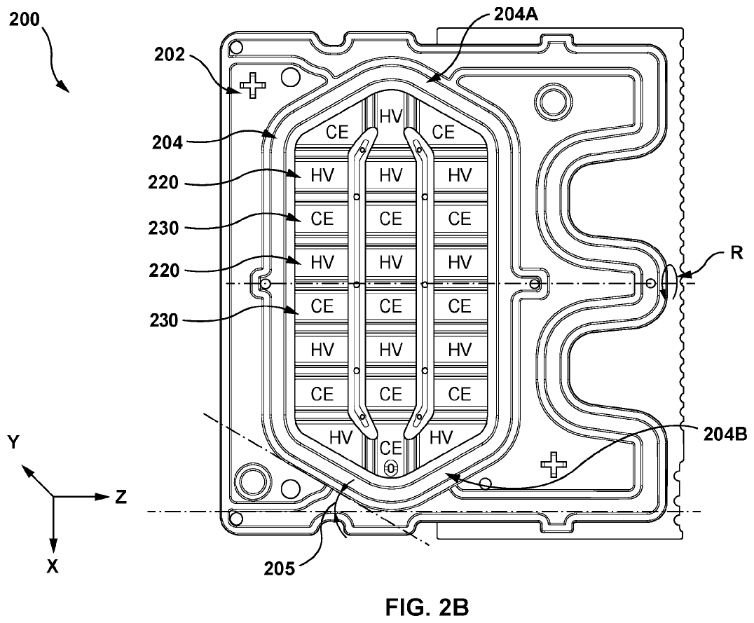

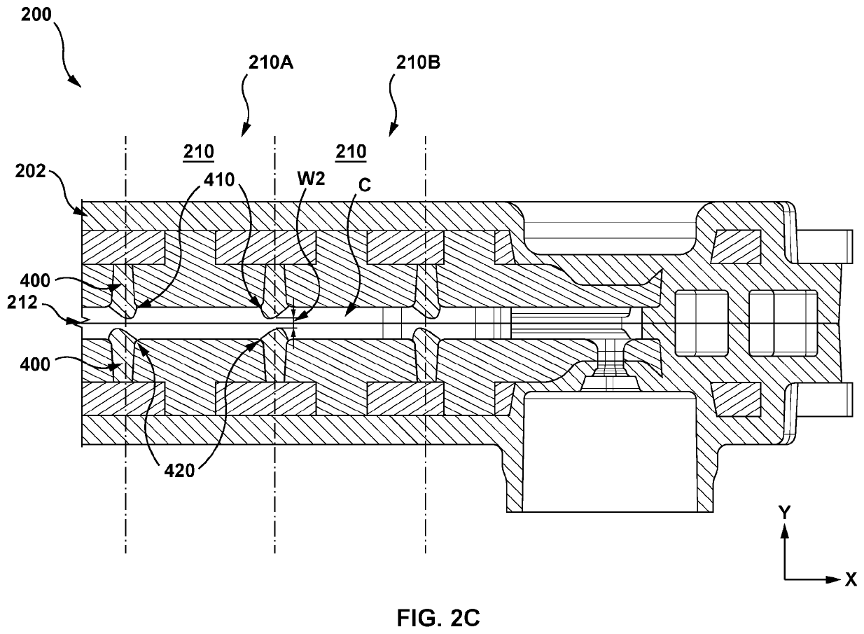

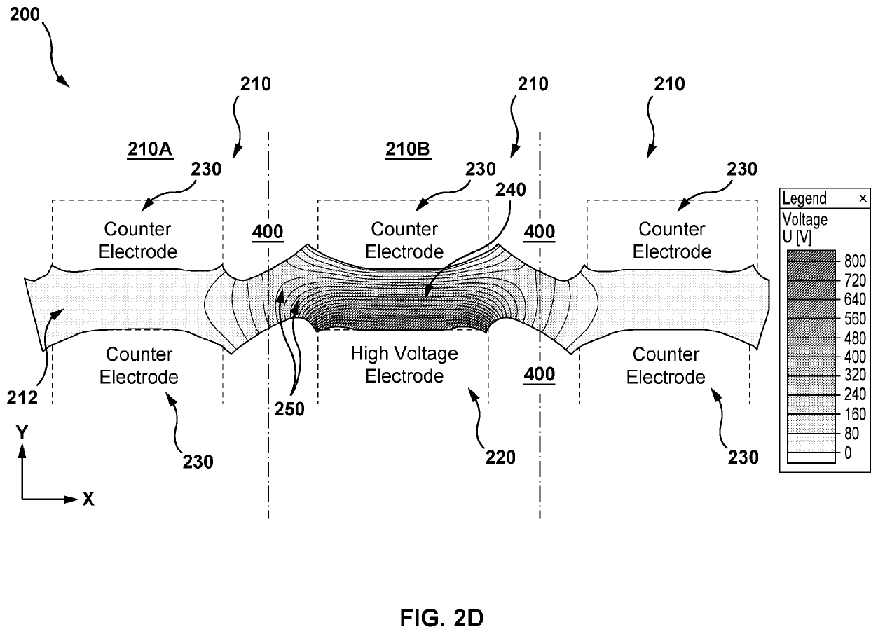

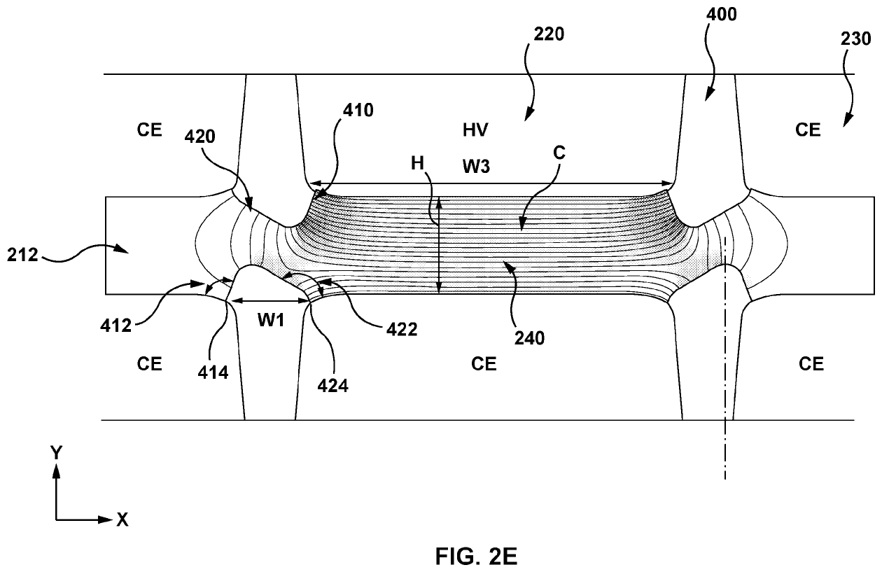

Exemplary embodiments of a device or cartridge 100/200 for the electroporation and/or transfection of at least one cell in a plurality of cells are shown in FIGS. 1A-2D. The device or cartridge 100/200 (also called electroporation cartridge herein) may be configured for insertion into, and electrical connection with, an electroporation device (not shown) configured for providing electrical energy to the device or cartridge 100/200 and generating the electrical pulses/electrical fields further described herein. The cartridge 100/200 is generally constructed to increase cell viability (i.e., survival rate of electroporated/transfected cells), cargo delivery (i.e., successful transfection of cells), number of transfected cells (i.e., total count of transfected cells post-electroporation), and transfection efficiency during the electroporation procedure in both small and large formats. The device generally comprises the cartridge 100/200 having a body 102/202 divided into a plurality of separate chambers 110/210, each chamber of the plurality of separate chambers 110/210 which are liquid-impermeable structures for the containment and flow therethrough of the aqueous solution or media comprising the at least one cell of the plurality of cells and/or the solution containing the external molecules for introduction into the at least one cell. At least one exchange path 112/212 runs through the body 102/202 of the device or cartridge 100/200 and fluidly connects adjacent chambers 110A/110B/210A/210B of the plurality of separate chambers 110/210 to allow the flowing media comprising the at least one cell of the plurality of cells to traverse through the plurality of separate chambers 110/210. The at least one exchange path 112/212 comprises at least one constriction 300/400 disposed therein for functionally separating adjacent chambers 110A/110B/210A/210B, but still allowing the flow of the media from one chamber 110A/210A to an adjacent chamber 110B/210B along the exchange path 112/212 fluidly connecting the plurality of separate chambers 110/210. As illustrated in FIGS. 1A and 2A, a plurality of constrictions 300/400 are disposed between adjacent chambers 110A/210A/110B/210B, the constrictions 300/400 which are described in greater detail herein with respect to their corresponding device 100/200. At least one high voltage electrode 120/220 may be disposed in some (suitably all) of the plurality of separate chambers 110/210, adjacent to the at least one constriction 300/400 of the at least one fluid exchange path 112/212. At least one counter electrode 130/230 may be disposed opposite the high voltage electrode 120/220, adjacent to the at least one constriction 300/400 of the at least one exchange path 112/212.

FIGS. 1A-1D illustrate a first embodiment of the device or cartridge 100 of the present invention. The cartridge 100 comprises a body 102 having an X-axis along its length (the X-axis being parallel to a fluid exchange path 112), a Z-axis along its width, and a Y-axis along its height. In embodiments, the body 102 of the cartridge may have a width (i.e., along the Z-axis) of approximately 60 to 80 millimeters (suitably 73.5 millimeters), and a height (i.e., along the X-axis) of approximately 70 to 90 millimeters (suitably 81.5 millimeters), as exemplified in FIG. 1A. In embodiments, the width of the body 102 of the cartridge 100 may be between 50-200 millimeters, and the height of the body 102 of the cartridge 100 may be between 50-200 millimeters. The width and the height of the body 102 of the cartridge 100 may vary in accordance with end user/laboratory specifications, and should not be interpreted as being limited to the embodiments described herein and shown in FIGS. 1A-1D. The body 102 is divided into a plurality of chambers 110 for the containment and flow therethrough of the media containing the at least one cell of the plurality of cells along the fluid exchange path 112 that fluidly connects adjacent chambers 110A, 110B of the plurality of chambers 110. In embodiments, the body 102 may be divided into twenty-four total chambers 110, equally divided into four rows of six chambers 110 along the Z-axis of the body 102, as exemplified in FIG. 1B. Each row of the plurality of chambers 110 may provide a capacity for containing a total volume of 0.5 milliliters of the media containing the at least one cell of the plurality of cells (i.e. across each chamber of the plurality of chambers 110), such that the total volume capacity of the four rows of plurality of chambers 110 (i.e., the volume capacity of the cartridge 100) is equivalent to at least 2 milliliters. In embodiments, each individual chamber of the plurality of chambers 110 has a capacity of at least 20 microliters, up to about 100 microliters, and preferably about 83 microliters. In embodiments, the volume capacity of a single row of chambers 110 may be between 0.25 to 2 milliliters. The total volume capacity of the four rows of chambers 110 may be between 1 to 8 milliliters depending on end user specifications. The volume capacity of each individual chamber 110, the rows of chambers 110, and or the overall cartridge 100 should not be interpreted as being limited to the embodiments described herein and presented in FIG. 1B.

Surrounding the plurality of chambers 110 on the body 102 is a perimeter 104 having a top perimeter portion 104A and a bottom perimeter portion 104B. The top perimeter portion 104A and the bottom perimeter portion 104B are defined by edges that are slightly angled with respect to the Z-axis, such that an angle 105 is formed relative to the Z-axis. In embodiments, the angle 105 may be between 5 to 30 degrees. More particularly, this angle 105 may be approximately 20 degrees. This angle 105 of the top and bottom portions 104A/104B of the perimeter 104 is formed in the body 102 of the cartridge 100 so as to prevent any trapping of the media containing the at least one cell in the corners of the plurality of chambers 110 adjacent to the perimeter, and/or to prevent the formation and trapping of bubbles in the corners of the plurality of chambers 110. The sloped face of this angle 105 further encourages a smooth flow of the media through the plurality of chambers 110 and the fluid exchange path 112, and prevents any unwanted stagnation.

Disposed within each chamber of the plurality of chambers 110 may be a high voltage electrode 120 and/or a counter electrode 130, configured for releasing/applying an electrical field or pulse to the media containing the at least one cell of the plurality of cells. The high voltage electrode 120 is configured to output a high voltage electric field, while the counter electrode 130 is configured to output an electric field at a voltage equivalent to or less than the high voltage electrode 120, and at an opposite polarity (i.e., positive vs. negative). In embodiments, the high voltage electrode 120 may be configured to output a high voltage electrode field having a voltage between 1 to 1200 volts; between 60 to 1200 volts; between 100 to 1100 volts; between 200 to 1000 volts; between 300 to 900 volts; between 400 to 800 volts; or between 500 to 700 volts. In embodiments, the plurality of chambers 110 includes a plurality of high voltage electrodes 120 and a plurality of counter electrodes 130, wherein the plurality of high voltage electrodes 120 and the plurality of counter electrodes 130 are alternately arranged within the cartridge within each chamber of the plurality of separate chambers 110. In embodiments, a chamber 110 comprises two counter electrodes 130 oppositely arranged along the Y-axis within the chamber 110, wherein the fluid exchange path 112 traverses between the two oppositely arranged electrodes. A chamber 110 may alternately comprise a high voltage electrode 120, and a counter electrode 130 oppositely arranged along the Y-axis within the chamber 110, wherein the fluid exchange path 112 traverses between the two oppositely arranged electrodes. It is possible to arrange some chambers 110 of the plurality of chambers 110 to comprise two counter electrodes 130 oppositely arranged along the Y-axis within the chamber 110, while the other remaining chambers 110 comprise a high voltage electrode 120, and a counter electrode 130 oppositely arranged along the Y-axis within the chamber 110. In chambers 110 comprising a high voltage electrode 120 and a counter electrode 130, the polarities of the high voltage electrode 120 and the counter electrode 130 may alternate, such that one of the high voltage electrode 120 and counter electrode 130 outputs a positive charge, while the other of the high voltage electrode 120 and counter electrode 130 outputs a negative charge.

Between adjacent chambers 110A, 110B of the plurality of chambers 110 is at least one constriction 300 disposed within the fluid exchange path 112, as shown in the circled portions of FIGS. 1A and 1D, for example. The at least one constriction 300 includes a non-conducting wall 304 which extends into the fluid exchange path 112 in a direction parallel to a planar surface of the body 102, or is formed along the Z-axis as illustrated in FIGS. 1A-1D. In this manner, a height H (i.e., along the Y-axis) of the plurality of chambers 110 and the fluid exchange path 112 is uniform along the X-axis of the cartridge 100. In embodiments, the height H of the at least one chamber 110 and/or the fluid exchange path 112 is at least 1.5 millimeters, as exemplified in FIG. 1C. In embodiments, the height H of the at least one chamber 110 and/or the fluid exchange path 112 may be between 0.5 to 2 millimeters, depending on end user specifications. The at least one constriction 300 further comprises a rounded protrusion 302 (see FIGS. 1A and 1D) extending from the non-conducting wall 304 and into the exchange path 112, such that the rounded protrusion 302 defines a channel C of the exchange path 112. In embodiments, the rounded protrusion has a radius of at least 0.5 millimeters. As shown in FIGS. 1A-1D, multiple constrictions 300 are arranged to be staggered between each chamber of the plurality of chambers 110, so as to functionally separate each chamber.

By incorporating the constrictions 300 into the exchange paths 112 and between each chamber 110, electrical field interference and pressure interference between adjacent chambers 110A/110B of the plurality of chambers 110 is restricted or limited by the rounded protrusions 302 of said constrictions 300. More particularly, no direct electrical field applied in a first chamber 110A via electrodes 120/130 may develop within a neighboring chamber 110B due to the placement of the constrictions 300 within the fluid exchange path 112 fluidly connecting said chambers 110A/110B. This functional separation of chambers 110 allows for the possibility to partially fill the device or cartridge 100 with media comprising the at least one cell, with respect to the cartridge's 100 full capacity. Alternatively, the functional separation of chambers 110 allows for identification of an optimal electrical pulse to be applied via the electrodes 120/130 in a single, isolated chamber 110, the optimal electrical pulse which may then be applied to the remaining chambers 110, resulting in greater electroporation and/or transfection efficiencies in large volume applications. Separating each chamber 110 with the constrictions 300 as described herein further results in a greater enhanced transfection performance.

FIGS. 2A-2C illustrate a second embodiment of the device or cartridge 200 of the present invention. The cartridge 200 comprises a body 202 having an X-axis along its length (the X-axis being parallel to a fluid exchange path 212), a Z-axis along its width, and a Y-axis along its height. In embodiments, the body 202 of the cartridge may have a width (i.e., along the Z-axis) of about 60 to 80 millimeters, suitably approximately 73.5 millimeters, and a height (i.e., along the X-axis) of approximately 70 to 90 millimeters, suitably about 81.5 millimeters, as exemplified in FIG. 2A. The width and the height of the body 202 of the cartridge 200 may vary in accordance with end user/laboratory specifications. In embodiments, the width of the body 202 of the cartridge 200 may be between 50-200 millimeters, and the height of the body 202 of the cartridge 200 may be between 50-200 millimeters. The width and the height of the body 202 of the cartridge 200 may vary in accordance with end user/laboratory specifications, and should not be interpreted as being limited to the embodiments described herein and shown in FIGS. 2A-2C. The body 202 is divided into a plurality of chambers 210 for the containment and flow therethrough of the media containing the at least one cell of the plurality of cells along the fluid exchange path 212 that fluidly connects adjacent chambers 210A, 210B of the plurality of chambers 210. In embodiments, the body 202 may be divided into twenty-four total chambers 210, equally divided into eight rows of three chambers 210 along the Z-axis of the body 202, as exemplified in FIG. 2A. Each row of the plurality of chambers 210 may provide a capacity for containing a volume of about 0.25 milliliters of the media containing the at least one cell of the plurality of cells across each chamber of the plurality of chambers, such that the total volume capacity of the eight rows of plurality of chambers 210 (i.e., the volume capacity of the cartridge 200) is equivalent to at least 2 milliliters. In embodiments, each separate chamber of the plurality of chambers 210 has a capacity of at least 20 microliters, up to and preferably about 83 microliters. In embodiments, the volume capacity of a single row of chambers 210 may be between 0.25 to 2 milliliters. The total volume capacity of the four rows of chambers 210 may be between 1 to 8 milliliters depending on end user specifications. The volume capacity of each individual chamber 210, the rows of chambers 210, and or the overall cartridge 200 should not be interpreted as being limited to the embodiments described herein and presented in FIG. 2A.

Surrounding the plurality of chambers 210 on the body 202 is a perimeter 204 having a top perimeter portion 204A and a bottom perimeter portion 204B. The top perimeter portion 204A and the bottom perimeter portion 204B are defined by edges that are slightly angled with respect to the Z-axis, such that an angle 205 is formed relative to the Z-axis. In embodiments, the angle 205 may be between 5 to 30 degrees. More particularly, this angle 205 may be approximately 20 degrees. This angle 205 of the top and bottom portions 204A/204B of the perimeter 204 is formed in the body 202 of the cartridge 200 so as to prevent any trapping of the media containing the at least one cell in the corners of the plurality of chambers 210 adjacent to the perimeter, and/or to prevent the formation and trapping of bubbles in the corners of the plurality of chambers 210. The sloped face of this angle 205 further encourages a smooth flow of the media through the plurality of chambers 210 and the fluid exchange path 212 and prevents any unwanted stagnation.

Disposed within each chamber of the plurality of chambers 210 may be a high voltage (HV) electrode 220 and/or a counter electrode (CE) 230, configured for releasing/applying an electrical field or pulse 240 to the media containing the at least one cell of the plurality of cells, as best illustrated in the diagrammatic views of FIGS. 2D and 2E. The high voltage electrode 220 is configured to output a high voltage electric field 240, while the counter electrode 230 is configured to output an electric field at a voltage equivalent to or less than the high voltage electrode 220, and at an opposite polarity (i.e., positive vs. negative). In embodiments, the high voltage electrode 220 may be configured to output a high voltage electrode field having a voltage between 1 to 1200 volts; between 60 to 1200 volts; between 100 to 1100 volts; between 200 to 1000 volts; between 300 to 900 volts; between 400 to 800 volts; or between 500 to 700 volts. In embodiments, the plurality of chambers 210 includes a plurality of high voltage electrodes 220 and a plurality of counter electrodes 230, wherein the plurality of high voltage electrodes 220 and the plurality of counter electrodes 230 are alternately arranged within the cartridge within each chamber of the plurality of separate chambers 210. In embodiments, a chamber 210 comprises two counter electrodes 230 oppositely arranged along the Y-axis within the chamber 210, wherein the fluid exchange path 212 traverses between the two oppositely arranged electrodes. A chamber 210 may alternately comprise a high voltage electrode 220, and a counter electrode 230 oppositely arranged along the Y-axis within the chamber 210, wherein the fluid exchange path 212 traverses between the two oppositely arranged electrodes. It is possible to arrange some chambers of the plurality of chambers 210 to comprise two counter electrodes 230 oppositely arranged along the Y-axis within the chamber 210, while the other remaining chambers 210 comprise a high voltage electrode 220, and a counter electrode 230 oppositely arranged along the Y-axis within the chamber 210.

FIG. 2B illustrates the arrangement of the electrodes 220/230 in one half of the body 202 of the device or cartridge 200, as if the body 202 were split across its planar surface (i.e., the Z-axis). Here, the electrodes 220/230 are arranged in a manner where the middle rows of the plurality of chambers 210 (i.e., the second to seventh rows) comprise alternating rows of all high voltage electrodes 220, and all counter electrodes 230, the top row of chambers 210 (i.e., the first row) comprises two counter electrodes 230 on outside chambers 210 staggering a high voltage electrode 220 on a middle chamber 210, and the bottom row of chambers 210 (i.e., the eighth row) comprises two high voltage electrodes 220 on outside chambers 210 staggering a counter electrode 230 on a middle chamber 210. From top to bottom (i.e., the three columns of eight chambers 210 formed along the X-axis), each chamber 210 alternates from high voltage electrodes 220 to counter electrodes 230 along the column. This pattern of alternatingly arranged high voltage electrodes 220 and counter electrodes 230 allow an identical arrangement of electrodes to be provided on a second half portion of the body 202 of the device or cassette 200 (identical to the first half depicted in FIG. 2B), such that by flipping this second half over a rotational axis R, the body 202 of the chamber 200 may be formed where each high voltage electrode 220 opposes a counter electrode 230 along the Y-axis in each chamber 210. In chambers 210 that comprise a high voltage electrode 220 and a counter electrode 230, such as those diagrammed in FIGS. 2D and 2E, the polarities of said high voltage electrode 220 and said counter electrode 230 may alternate, such that one of the high voltage electrode 220 and counter electrode 230 outputs a positive charge, while the other of the high voltage electrode 220 and counter electrode 230 outputs a negative charge.

Between adjacent chambers 210A, 210B of the plurality of chambers 210 is at least one constriction 400 disposed within the fluid exchange path 212, as shown in the circled portion of FIG. 2A, and further detailed in the cross-section of FIG. 2C. As shown in FIG. 2C, a plurality of constrictions 400 are disposed along the fluid exchange path 212, between each chamber of the plurality of chambers 210. The at least one constriction 400 extends into the fluid exchange path 212 in a direction perpendicular to a planar surface of the body 202, or is formed along the Y-axis as illustrated in FIG. 2C, and diagrammed in FIGS. 2D-2E. Put another way, the at least one constriction 400 extends orthogonal to the flow of the media between the electrodes 220/230. The at least one constriction 400 may comprise a width W1 formed between a first endpoint 414 of the first edge 410, and a second endpoint 424 of the second edge 420 (see FIG. 2E). In embodiments, the width W1 of the at least one constriction 400 may be at least 1.5 millimeters. A second constriction 400 is disposed within the fluid exchange path 212 along the Y-axis and opposite the first constriction 400. In this arrangement, a separation distance W2 formed between the tip of the first constriction (i.e., where the first edge 410 and the second edge 420 meet) and the tip of the second constriction is at least 0.5 millimeters (see FIG. 2C).

In embodiments, the height H of the at least one chamber 210 between each constriction 300 is at least 1.5 millimeters. In embodiments, the height H of the at least one chamber 210 and/or the fluid exchange path 212 may be between 0.5 to 2 millimeters, depending on end user specifications. The constrictions 400 each comprise a first edge 410 and a second edge 420, wherein the first edges 410 define a chamber width W3, and a second edges 420 define a channel C of the fluid exchange path 212, as illustrated in FIG. 2E. A first angle 412 formed by the first edge 410 and an adjacent high voltage electrode 220 or counter electrode 230 is approximately 60-120 degrees, 70-110 degrees, 80-100 degrees, or approximately 90 degrees. A second angle 422 formed by the second edge 420 and an adjacent high voltage electrode 220 or counter electrode 230 is approximately 60-120 degrees, 70-110 degrees, 80-100 degrees, or approximately 90 degrees. The first edges 410 of the constrictions 400 are formed and angled in this manner so as to restrict electric field interference between adjacent chambers 210A/210B during electroporation procedures, as illustrated in FIG. 2D, where the higher-voltages are shown to be restricted within the individual chamber 210, as diagrammed by equi-potential lines 250 (the voltage ranges of which are described by the legend provided in FIG. 2D). The second edges 420 of the constrictions 400 are formed and angled in this manner so as to prevent turbulence and/or cell stress during flow of the media comprising the at least one cell through the fluid exchange path 212 (and through each constriction 400 and associated chamber 210).

By incorporating the constrictions 400 into the exchange paths 212 between each chamber 210, electrical field interference and pressure interference between adjacent chambers 210A/210B of the plurality of chambers 210 is restricted by the edges 410/420 of said constrictions 400. More particularly, no direct electrical field applied in a first chamber 210A via electrodes 220/230 may develop within a neighboring chamber 210B due to the placement of the constrictions 400 within the fluid exchange path 212 fluidly connecting said chambers 210A/210B. This functional separation of chambers 210 allows for the possibility to partially fill the device or cartridge 200 with media comprising the at least one cell, with respect to the cartridge's 200 full capacity. Alternatively, the functional separation of chambers 210 allows for identification of an optimal electrical pulse to be applied via the electrodes 220/230 in a single, isolated chamber 210, the optimal electrical pulse which may then be applied to the remaining chambers 210, resulting in greater electroporation and/or transfection efficiencies in large volume applications. Separating each chamber 210 with the constrictions 400 as described herein further results in a greater enhanced transfection performance.

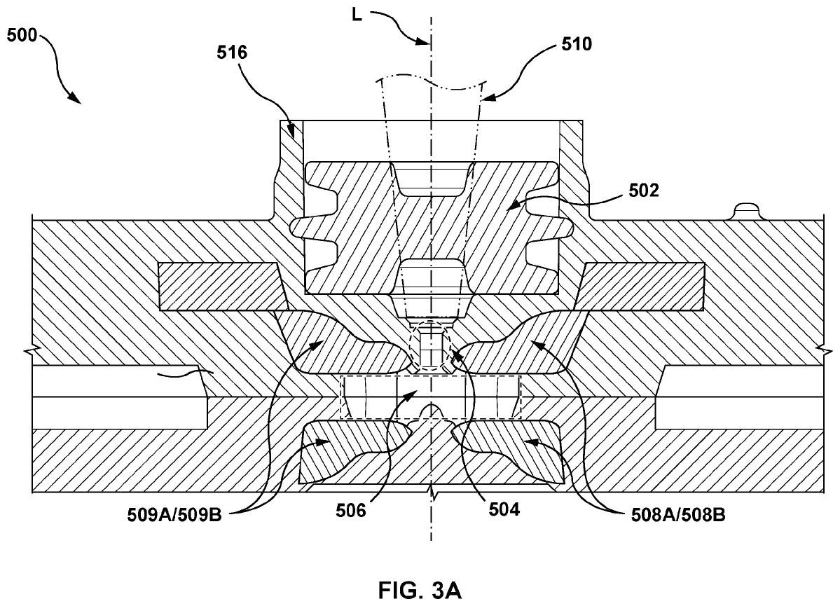

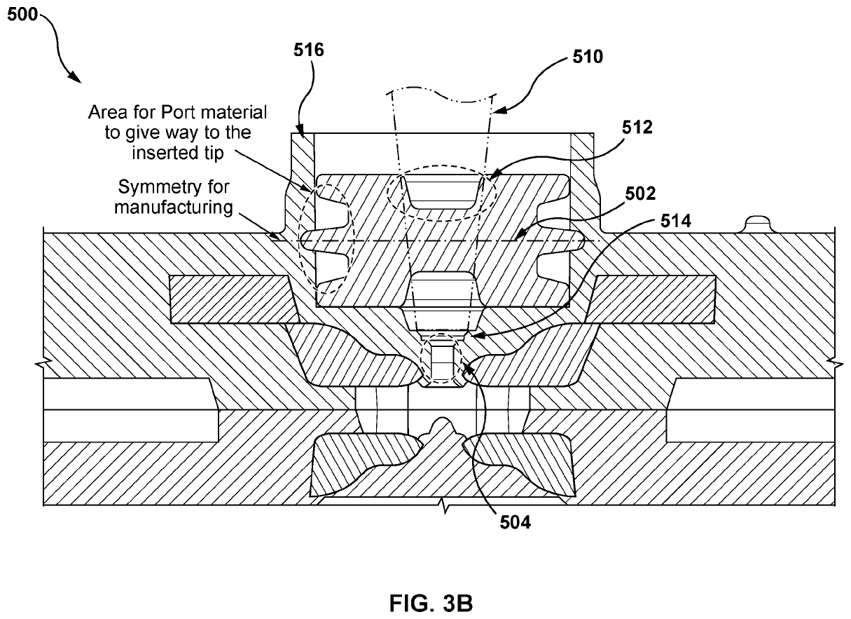

As illustrated in FIGS. 3A and 3B, the device or cartridge 100/200 may further comprise an inlet 500 disposed at an inflow end of the at least one fluid exchange path 112/212. In the embodiments of the device or cartridge 100/200, the fluid exchange paths 112/212 meet at the inlet 500, the inlet 500 which serves as the sole dedicated port for supply of the media comprising the at least one cell as described herein. A corresponding outlet (not shown) may be disposed at an outflow end of the at least one fluid exchange path 112/212. In the embodiments of the device or cartridge 100/200, the fluid exchange paths 112/212 meet at the outlet (not shown), which serves as the sole egress for media comprising the electroporated and/or transfected cells.

The inlet 500 is constructed so as to minimize the dead volume of media/cell suspension not reached by the electrical field output of the electrodes 120/130/220/230 during the electroporation procedures. The inlet 500 comprises an injection inlet 502, a dead volume area 504, an electrically active area 506, and at least one inlet electrode pair 508A/508B/509A/509B. More particularly, the injection inlet 502 may be a pre-slit inlet that includes an insertion guide 512 disposed within the body 102/202 of the cartridge 100/200 near the entry of the injection port 502 for receiving a dispenser 510 therein and for facilitating insertion of said dispenser 510 into said inlet 500. As used herein “dispenser” refers to any suitable device or structure for providing a media/cell suspension, including for example, a needle, a syringe, a pipette, a micropipette, various tubing, etc. The pre-slit configuration of the injection inlet 502 allows for the material that makes up the injection inlet 502 (e.g., rubber) to spread outwards, give way, or expand to the dispenser 510 as it is inserted into the inlet 500. The pre-slit configuration may further assist in guiding the dispenser 510 through the inlet 500. The injection inlet 502 is further presented in an oversized triple seal design, which ensures leak tightness of the area of the inlet 500 inside the cartridge 100/200. The oversizing of this injection inlet 502 further ensures the pre-slit portion stays pressed closed when not interacting with the dispenser 510. In embodiments, the dispenser 510 may be a pipette tip, a needle, a tube, or any other suitable device for delivery of the media containing the at least one cell of the plurality of cells. Media/cell suspensions introduced into the injection inlet 502 flow into the dead volume area 504, which serves as a narrow passageway between the injection port 502 and the electrically active area 506 for the media comprising the at least one cell of the plurality of cells to flow therethrough. An end stop 514 is disposed at the end of the insertion guide 512 and adjacent to the dead volume area 504, and is configured for receiving the end of the dispenser 510 during the procedure of insertion of the dispenser 510 into the inlet 500. Disposed on either side of, and surrounding the electrically active area 506, are pairs of inlet electrodes 508A/508B/509A/509B, arranged such that the first pair of inlet electrodes 508A/508B oppose the electrically active area with respect to a vertical axis L of the inlet 500, and the second pair of inlet electrodes 509A/509B oppose the electrically active area with respect to the vertical axis L of the inlet. In embodiments, the pairs of inlet electrodes 508A/508B/509A/509B comprise high voltage electrodes and counter electrodes. More particularly, one of the first pair of inlet electrodes 508A/508B may be a high voltage electrode, while the other of the first pair of inlet electrodes 508A/508B may be a counter electrode. One of the second pair of inlet electrodes 509A/509B may be a high voltage electrode, while the other of the second pair of inlet electrodes 509A/509B may be a counter electrode. The injection inlet 502, dead volume area 504, electrically active area 506, insertion guide 512, and at least one inlet electrode pair 508A/508B/509A/509B of the inlet 500 are formed within the body 102/202 of the device or cassette 100/200 such that the inlet 500 is symmetrical along its vertical axis L. The symmetrical orientation of the inlet 500 allows for simplification of manufacturing of the inlet 500 into the device or cartridge 100/200 described herein, as illustrated in FIG. 3B. The inlet 500 may be held in position by thermoforming the rim 516 above the injection inlet 502, which ensures the resilient material of the injection inlet 502 stays within the inlet 500 and is not accidentally removed upon extraction of the dispenser 510.

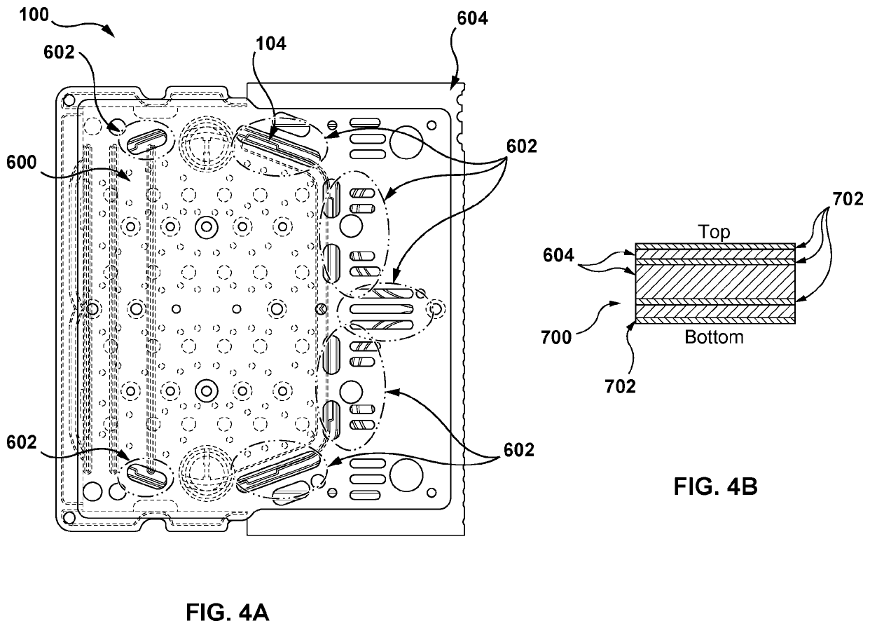

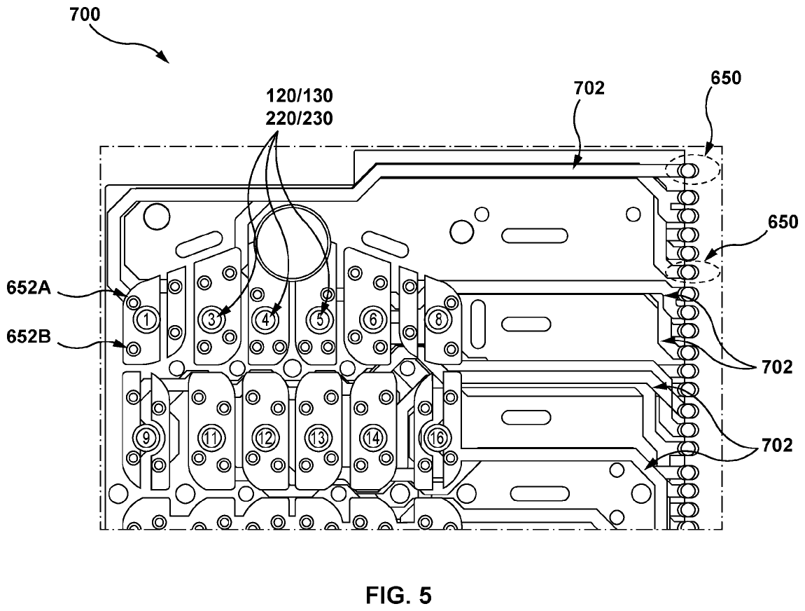

FIGS. 4A and 4B illustrate an overmolded circuit board 600 integrated with the device or cartridge 100 as previously described herein, while FIG. 5 illustrates a bare circuit board 700 as described in greater detail below (i.e., prior to overmolding). As shown in FIG. 4A, ultraviolet (UV) light cutouts 602 are spatially disposed in areas around or adjacent to the perimeter 104 that surrounds the plurality of chambers 110 as previously described herein. When forming the device or cartridge 100, a mold 604 (as shown in the cross-section of FIG. 4B) is formed over or around layers of a conductive copper substrate 702 of the circuit board 700 (the circuit board 700 which is further detailed in FIG. 5) and secured to the circuit board 700 via UV-activated adhesive to form the overmolded circuit board 600. The UV light cutouts 602 thus allow for proper entry of UV light for the curing of said UV-activated adhesive, once the overmolded circuit board 600 is formed. Implementation of the UV light cutouts 602 further achieves a reduction in deformation and mechanical tension on the circuit board 700 and its relative components. In embodiments, the mold may be an overlaying layer of plastic, carbon, glass, polymer, or any other suitable material. A similar overmolded circuit board may be formed for integration with the device or cartridge 200 described herein.

FIG. 5 illustrates the circuit board 700 in a side elevational view. The circuit board 700 comprises electrical interfaces 650 disposed adjacent to an edge of the circuit board 700, and multiple electrical paths 652A/652B for establishing a redundant connection to the electrodes 120/130/220/230, wherein the electrical interfaces 650 are in electrical communication with the multiple electrical paths 652A/652B. In embodiments, the circuit board 700 may be a multilayer printed circuit board (PCB), as shown in FIG. 5. In embodiments, the circuit board 700 may be a single-sided PCB, a double-sided PCB, a rigid PCB, or a flex PCB. By establishing the redundant connection to the electrodes 120/130/220/230 via the multiple electrical paths 652A/652B, the electrical interface of the electrodes 120/130/220/230 to the overmolded circuit board 600 may be measured, and may further allow for in-process quality checks. This redundant connection further allows the overmolded metals (i.e., the conductive materials) of the circuit board 700 to be designed so that both the electrical interfaces 650 and the contact points of the electrical paths 652A/652B with the electrodes 120/130/220/230 allow for measurement of conductivity of said electrodes 120/130/220/230 during the manufacturing process to ensure robust electrode manufacturing and functionality.

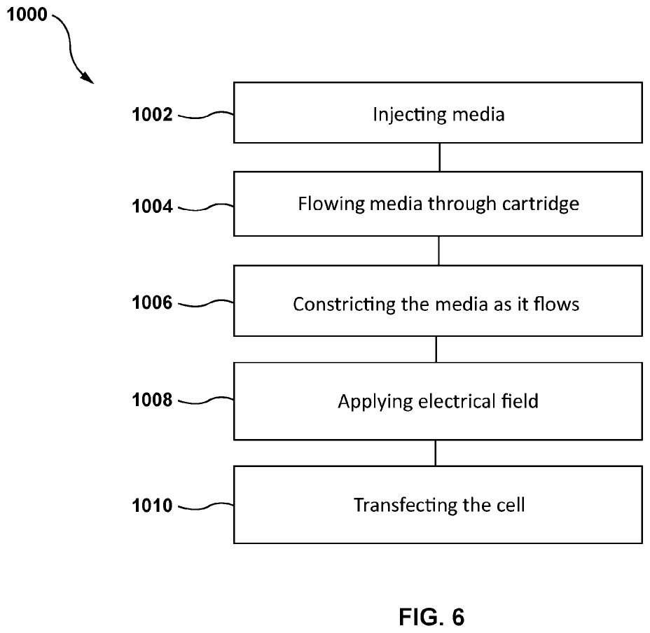

Further described are systems and methods of electroporating and/or transfecting a cell using the device or cartridge 100/200 as described herein. FIG. 6 illustrates an exemplary method 1000 of electroporating at least one cell in a plurality of cells. In a first step 1002, a media containing the at least one cell is injected into the device or cartridge 100/200. The step 1002 may include injecting the media through the inlet 500 of the cartridge 100/200. More particularly, step 1002 may include inserting a dispenser 510 into the injection inlet 502, the injection inlet 502 which may be pre-slit so as to more easily receive the dispenser 510. Inserting the dispenser 510 may include guiding the dispenser through the injection inlet 502 via the insertion guide 512, and stopping traversal of the dispenser 510 through the injection inlet 502 via the end stop 514. Inserting the dispenser 510 may include outputting an end stop response upon contacting the end stop 514 with the dispenser 510, such as a haptic feedback (i.e., vibration), an audible indicator, or a flashing/blinking light, to indicate that insertion of the dispenser 510 into the inlet 500 is completed. Once the dispenser 510 is inserted, the media containing the at least one cell of the plurality of cells mays be introduced, flowed, or otherwise injected into the device or cartridge 100/200.

A next step 1004 of the method 1000 comprises flowing the media containing the at least one cell of the plurality of cells through the plurality of separate chambers 110/210 of the cartridge 100/200. More particularly, step 1004 may include flowing the media through the at least one exchange path 112/212 which fluidly connects the adjacent chambers 110A/110B/210A/210B of the plurality of separate chambers 110/210. The flow of the media through the plurality of chambers 110/210 and/or the at least one fluid exchange path 112/212 may be generated by the force of the dispenser 510 introducing, flowing, or otherwise injecting them media into the device or cartridge 100/200 through the inlet 500, as previously described herein. In embodiments, the flow of the media through the plurality of chambers 110/210 and/or the at least one fluid exchange path 112/212 may be generated via repetitive filling procedures, wherein the dispenser 510 is a tubing connected to the inlet 500, and the dispenser 510 continually pumps media into the device or cartridge 100/200 from an outside source (i.e., reservoirs or bags containing the media comprising the at least one cell of the plurality of cells). In the repetitive filling procedure, a separate clearing pump (not shown) is used to push air into the cartridge 100/200 after each cycle of electroporation/transfection to ensure the media comprising the post-transfected cells egress through the outlet and effectively empty the cartridge 100/200. The dispenser 510 may then automatically fill the cartridge 100/200 with new media comprising at least one cell for electroporation and/or transfection.

A next step 1006 of the method 1000 comprises constricting the media as it flows through the plurality of chambers 110/210 and the at least one exchange path 112/212 via at least one constriction 300/400 disposed within the at least one exchange path 112/212. The flow of the media through the plurality of chambers 110/210 and/or the at least one fluid exchange path 112/212 may be generated by the force of the dispenser 510 introducing, flowing, or otherwise injecting the media into the device or cartridge 100/200 through the inlet 500, as previously described herein. The flow of the media through the plurality of chambers 110/210 and/or the at least one exchange path 112/212 is not impacted by the at least one constriction 300/400 disposed within the fluid exchange path 112/212 due to the slow-moving nature of the media as it flows therethrough. The step 1006 may include preventing turbulence and/or cell stress during flow of the media comprising the at least one cell through the fluid exchange path 112/212 and through each constriction 300/400 and associated chamber 110/210 by flowing the media over the rounded protrusion 302 or the first/second edges 410/420 of the constrictions 300/400, respectively.

A next step 1008 of the method 1000 comprises applying an electrical field to the at least one cell within the media via the at least one high voltage electrode 120/220 disposed in each chamber of the plurality of separate chambers 110/210, and via the at least one counter electrode 130/230 disposed opposite the at least one high voltage electrode 120/220 as previously described herein. Application of the electrical field to the at least one cell results in the electroporation of the at least one cell, where the pores of the cell membrane are opened and transfection is subsequently achievable. More particularly, the electrical field applied to the at least one cell may be between 1 to 1200 Volts. In embodiments, the high voltage electrode 120/220 may be configured to output a high voltage electrode field having a voltage between 60 to 1200 volts; between 100 to 1100 volts; between 200 to 1000 volts; between 300 to 900 volts; between 400 to 800 volts; or between 500 to 700 volts. The electrical field applied to the at least one cell may be done so in a multi-pulsed manner, wherein a plurality of electrical field pulses are applied to the at least one cell. Step 1008 may include applying a polarly opposite electrical field to the at least one cell via the at least one counter electrode 130/230 disposed within the chambers 110/210. In embodiments, a resistance measurement may be obtained prior to the step 1008 of applying the electric field, where the resistance measurement may be used to help detect incompletely filled chambers 110/210. Incompletely filled chambers 110/210 can favor the generation of arc discharges, or electrical discharge of relatively high currents at relatively low voltages, especially for outputs measured at above 800 Volts. Arc discharges may lead to potentially incomplete delivery of the electrical field from the electrodes 120/130/220/230, which may potentially increase damage to the at least one cell to be electroporated and/or transfected. Clipping the voltage of the applied electrical fields (i.e., capping the voltage output so that it does not exceed a predetermined threshold) may be utilized to reduce the risk of such arc discharges.

In embodiments, method 1000 further includes transfecting the cell with one or more molecules (i.e. molecules such as dye, or nucleic acids, or biologically active molecules, including oligonucleotides, or peptides) such that the molecules enter the cell. Suitably the cells are transfected with a nucleic acid that enters the cell via the electroporation and then is trafficked to the nucleus. Transfection of one or more molecules, suitably biologically active molecules, in general does not require any additional treatment, delivery construct, or encapsulation of the molecules. However, in embodiments, a viral vector can be utilized that carries desirable nucleic acids that can then be transfected into the cells. In other embodiments, a lipid-based or polymer-based carrier (e.g., liposome, micelle, polymer sphere, dendrimer, or other transfection reagents etc.) can also be utilized to introduce the molecules, such as proteins or nucleic acids. In other embodiments, free nucleic acids can be used, or in embodiments, chemically modified nucleic acids can be used that have increased stability (e.g., methylation, etc.).

The foregoing description has been presented for purposes of illustration and enablement and is not intended to be exhaustive or to limit the invention to the precise form disclosed. Other modifications and variations are possible in light of the above teachings. The embodiments and examples were chosen and described in order to best explain the principles of the invention and its practical application and to thereby enable others skilled in the art to best utilize the invention in various embodiments and various modifications as are suited to the particular use contemplated. It is intended that the appended claims be construed to include other alternative embodiments of the invention.

Embodiments of the present disclosure include the following examples.

Embodiment 1 includes a device for electroporating at least one cell in a plurality of cells, comprising: a cartridge divided into a plurality of separate chambers; at least one exchange path fluidly connecting adjacent chambers of the plurality of separate chambers, the at least one exchange path having a constriction disposed therein functionally separating adjacent chambers; at least one high voltage electrode disposed in each chamber of the plurality of separate chambers and adjacent to the constriction of the at least one exchange path; at least one counter electrode disposed opposite the high voltage electrode and adjacent to the constriction of the at least one exchange path; and an inlet.

Embodiment 2 includes the device of embodiment 1, wherein the inlet is disposed at an end of the at least one exchange path, the inlet having an injection inlet, a dead volume area, an electrically active area, and at least one inlet electrode pair consisting of a high voltage electrode and a counter electrode disposed therein.

Embodiment 3 includes the device of embodiment 2, wherein the injection inlet further includes an insertion guide for receiving a dispenser into the inlet.

Embodiment 4 includes the device of embodiment 1, wherein a first constriction of the at least one exchange path is formed along a Y-axis of the cartridge, the first constriction having a first edge and a second edge, the first edge defining a chamber width, the second edge defining a channel of the exchange path.

Embodiment 5 includes the device of embodiment 4, wherein an electric field interference between the adjacent chambers is restricted by the first edge.

Embodiment 6 includes the device of embodiment 4, wherein a first angle formed by the first edge and the high voltage electrode or the counter electrode is approximately 60-120 degrees, and a second angle formed by the second edge and the high voltage electrode or the counter electrode is approximately 60-120 degrees.

Embodiment 7 includes the device of embodiment 4, wherein a width of the first constriction from a first endpoint of the first edge to a second endpoint of the second edge is at least 1.5 millimeters. Embodiment 8 includes the device of embodiment 4 further including a second constriction disposed within the at least one exchange path along the Y-axis and opposite the first constriction, such that the channel has a separation distance of at least 0.5 millimeters between the first constriction and the second constriction.

Embodiment 9 includes the device of embodiment 4 wherein a size of the at least one chamber is at least 1.5 millimeters in the Y-axis.

Embodiment 10 includes the device of embodiment 1, wherein the constriction of the at least one exchange path is formed along a Z-axis of the cartridge, the constriction having a rounded protrusion extending into the exchange path and defining a channel of the exchange path, the rounded protrusion having a radius of at least 0.5 millimeters.

Embodiment 11 includes the device of embodiment 10, wherein an electric field interference between the adjacent chambers is restricted by the rounded protrusion.

Embodiment 12 includes the device of embodiment 10, wherein a height of the at least one chamber and the at least one exchange path along the Y-axis is uniform along an X-axis of the cartridge.

Embodiment 13 includes the device of embodiment 12, wherein the height of the at least one chamber and the at least one exchange path is at least 1.5 millimeters.

Embodiment 14 includes the device of embodiment 1, wherein the at least one high voltage electrode and the at least one counter electrode are of alternating polarities.

Embodiment 15 includes the device of embodiment 1, further including a plurality of high voltage electrodes and a plurality of counter electrodes, wherein the plurality of high voltage electrodes and the plurality of counter electrodes are alternately arranged within the cartridge within each chamber of the plurality of separate chambers.

Embodiment 16 includes the device of embodiment 1, wherein the inlet is symmetrical along a vertical axis.

Embodiment 17 includes the device of embodiment 1, wherein each chamber of the plurality of separate chambers has a capacity of at least 20 microliters.

Embodiment 18 includes the device of embodiment 1, wherein a capacity of the cartridge is at least two (2) milliliters.

Embodiment 19 includes the device of embodiment 3, further including an end stop disposed between the injection inlet and the dead volume area for receiving the end of the pipette tip.

Embodiment 20 includes the device of embodiment 1, wherein a top perimeter and a bottom perimeter of the device surrounding the plurality of chambers are defined by edges having at least a 20 degree slope.

Embodiment 21 includes A method of electroporating at least one cell in a plurality of cells, comprising: injecting a media containing the at least one cell through an inlet of a cartridge, wherein the cartridge is divided into a plurality of separate chambers; flowing the media through the plurality of separate chambers of the cartridge, including flowing the media through at least one exchange path fluidly connecting adjacent chambers of the plurality of separate chambers; constricting the media as it flows through the at least one exchange path via a constriction disposed within the at least one exchange path, the constriction functionally separating the adjacent chambers; applying an electrical field to the at least one cell via at least one high voltage electrode disposed in each chamber of the plurality of separate chambers and adjacent to the constriction of the at least one exchange path, and via at least one counter electrode disposed adjacent to the constriction of the at least one exchange path and opposite the at least one high voltage electrode; and electroporating the at least one cell.

Embodiment 22 includes the method of embodiment 21, further including applying a polarly opposite electrical field to the at least one cell via the at least one counter electrode.

Embodiment 23 includes the method of embodiment 22, wherein a first constriction of the at least one exchange path is formed along a Y-axis of the cartridge, the first constriction further having a first edge and a second edge, the first edge defining a chamber width, the second edge defining a channel of the exchange path for constricting the media as it flows through the at least one exchange path.

Embodiment 24 includes the method of embodiment 23, further including the step of restricting an electric field interference between the adjacent chambers via the first edge of the first constriction.

Embodiment 25 includes the method of embodiment 21, further including a second constriction disposed within the at least one exchange path along the Y-axis and opposite from the first constriction, such that the channel has a separation distance of at least 0.5 millimeters provided between the first constriction and the second constriction within the at least one exchange path.

Embodiment 26 includes the method of embodiment 21, wherein the constriction of the at least one exchange path is formed along a Z-axis of the cartridge, the constriction further having a rounded protrusion defining a channel of the exchange path for constricting the media as it flows through the at least one exchange path, the rounded protrusion having a radius of at least 0.5 millimeters.

Embodiment 27 includes the method of embodiment 26, further including the step of restricting an electric field interference between the adjacent chambers via the rounded protrusion of the constriction.

Embodiment 28 includes the method of embodiment 21, wherein the inlet is disposed at an end of the at least one exchange path, and the inlet further includes an injection inlet having an insertion guide, a dead volume area, and an electrically active area, and the injecting a media containing the at least one cell through the inlet of a cartridge further includes: inserting a pipette tip into the insertion guide of the injection inlet to facilitate proper delivery of the pipette tip into the inlet; and receiving the end of the pipette tip via an end stop disposed between the injection inlet and the dead volume area.

Embodiment 29 includes the method of embodiment 28, further including: providing an end stop response when the step of inserting the pipette tip into the injection inlet is complete.

Embodiment 30 includes the method of embodiment 21 wherein the electrical field applied to the at least one cell via the at least one high voltage electrode and/or the at least one counter electrode is between 1 to 1200 Volts.

Embodiment 31 includes A method of transfecting at least one cell in a plurality of cells, comprising: injecting a media containing the at least one cell and at least one nucleic acid through an inlet of a cartridge, wherein the cartridge is divided into a plurality of separate chambers; flowing the media through the plurality of separate chambers of the cartridge, including flowing the media through at least one exchange path fluidly connecting adjacent chambers of the plurality of separate chambers; constricting the media as it flows through the at least one exchange path via a constriction disposed within the at least one exchange path, the constriction functionally separating the adjacent chambers; applying an electrical field to the at least one cell via at least one high voltage electrode disposed in each chamber of the plurality of separate chambers and adjacent to the constriction of the at least one exchange path, and at least one counter electrode disposed adjacent to the constriction of the at least one exchange path and opposite the high voltage electrode; and transfecting the at least one nucleic acid into the at least one cell.

Embodiment 32 includes the method of embodiment 31, further including applying a polarly opposite electrical field to the at least one cell via the at least one counter electrode.

Embodiment 33 includes the method of embodiment 32, wherein a first constriction of the at least one exchange path is formed along a Y-axis of the cartridge, the first constriction further having a first edge and a second edge, the first edge defining a chamber width, the second edge defining a channel of the exchange path for constricting the media as it flows through the at least one exchange path.

Embodiment 34 includes the method of embodiment 33, further including the step of restricting an electric field interference between the adjacent chambers via the first edge of the first constriction.

Embodiment 35 includes the method of embodiment 31, further including a second constriction disposed within the at least one exchange path along the Y-axis and opposite from the first constriction, such that the channel has a separation distance of at least 0.5 millimeters provided between the first constriction and the second constriction within the at least one exchange path.

Embodiment 36 includes the method of embodiment 31, wherein the constriction of the at least one exchange path is formed along a Z-axis of the cartridge, the constriction further having a rounded protrusion defining a channel of the exchange path for constricting the media as it flows through the at least one exchange path, the rounded protrusion having a radius of at least 0.5 millimeters.

Embodiment 37 includes the method of embodiment 36, further including the step of restricting an electric field interference between the adjacent chambers via the rounded protrusion of the constriction.

Embodiment 38 includes the method of embodiment 31, wherein the inlet is disposed at an end of the at least one exchange path, and the inlet further includes an injection inlet having an insertion guide, a dead volume area, and an electrically active area, and the injecting a media containing the at least one cell and at least one nucleic acid through the inlet of a cartridge further includes: inserting a pipette tip into the insertion guide of the injection inlet to facilitate proper delivery of the pipette tip into the inlet; and receiving the end of the pipette tip via an end stop disposed between the injection inlet and the dead volume area.

Embodiment 39 includes the method of embodiment 38, further including: providing an end stop response when the step of inserting the pipette tip into the injection inlet is complete.

Embodiment 40 includes the method of embodiment 31 wherein the electrical field applied to the at least one cell via the at least one high voltage electrode and/or the at least one counter electrode is between 1 to 1200 Volts.

Embodiment 41 includes a system for electroporating at least one cell in a plurality of cells, comprising: a cartridge divided into a plurality of separate chambers, each chamber of the plurality of separate chambers is configured to hold at least one cell and is fluidly connected via at least one exchange path, the at least one exchange path having a constriction disposed therein; at least one high voltage electrode disposed in said each chamber of the plurality of separate chambers and adjacent to the constriction of the at least one exchange path; at least one counter electrode disposed opposite the high voltage electrode and adjacent to the constriction of the at least one exchange path; an inlet disposed at an end of the at least one exchange path, the inlet having an injection inlet, a dead volume area, an electrically active area, and at least one inlet electrode pair consisting of a high voltage electrode and a counter electrode disposed therein; and an electroporation device configured to receive the cartridge and generate an electric pulse via the high voltage electrode and the counter electrode.

Embodiment 42 includes the system of embodiment 41, wherein the injection inlet further includes an insertion guide for receiving a dispenser into the inlet.

Embodiment 43 includes the system of embodiment 41, wherein a first constriction of the at least one exchange path is formed along a Y-axis of the cartridge, the first constriction having a first edge and a second edge, the first edge defining a chamber width, the second edge defining a channel of the exchange path.

Embodiment 44 includes the system of embodiment 43, wherein an electric field interference between the adjacent chambers is restricted by the first edge.

Embodiment 45 includes the system of embodiment 43, wherein a first angle formed by the first edge and the high voltage electrode or the counter electrode is approximately 60-120 degrees, and a second angle formed by the second edge and the high voltage electrode or the counter electrode is approximately 60-120 degrees.

Embodiment 46 includes the system of embodiment 43, wherein a width of the first constriction from a first endpoint of the first edge to a second endpoint of the second edge is at least 1.5 millimeters.

Embodiment 47 includes the system of embodiment 43, further including a second constriction disposed within the at least one exchange path along the Y-axis and opposite the first constriction, such that the channel has a separation distance of at least 0.5 millimeters between the first constriction and the second constriction.

Embodiment 48 includes the system of embodiment 43 wherein a size of the at least one chamber is at least 1.5 millimeters along the Y-axis.

Embodiment 49 includes the system of embodiment 41, wherein the constriction of the at least one exchange path is formed along a Z-axis of the cartridge, the constriction having a rounded protrusion extending into the exchange path and defining a channel of the exchange path, the rounded protrusion having a radius of at least 0.5 millimeters.

Embodiment 50 includes the system of embodiment 49, wherein an electric field interference between the adjacent chambers is restricted by the rounded protrusion.

Embodiment 51 includes the system of embodiment 49, wherein a height of the at least one chamber and the at least one exchange path along the Y-axis is uniform along an X-axis of the cartridge.

Embodiment 52 includes the system of embodiment 51, wherein the height of the at least one chamber and the at least one exchange path is at least 1.5 millimeters.

Embodiment 53 includes the system of embodiment 41, wherein the at least one high voltage electrode and the at least one counter electrode are of alternating polarities.

Embodiment 54 includes the system of embodiment 41, further including a plurality of high voltage electrodes and a plurality of counter electrodes, wherein the plurality of high voltage electrodes and the plurality of counter electrodes are alternately arranged within the cartridge within each chamber of the plurality of separate chambers.

Embodiment 55 includes the system of embodiment 41, wherein the inlet is symmetrical along a vertical axis.

Embodiment 56 includes the system of embodiment 41, wherein said each chamber of the plurality of separate chambers has a capacity of at least 20 microliters per chamber.

Embodiment 57 includes the system of embodiment 41, wherein a capacity of the cartridge is at least two (2) milliliters.

Embodiment 58 includes the system of embodiment 41, further including an end stop disposed between the injection inlet and the dead volume area for receiving the end of the pipette tip.

Embodiment 59 includes the system of embodiment 41, wherein a top perimeter and a bottom perimeter of the device surrounding the plurality of chambers are defined by edges having at least a 20 degree slope.

1. A device for electroporating at least one cell in a plurality of cells, comprising:
a cartridge divided into a plurality of separate chambers;
at least one exchange path fluidly connecting adjacent chambers of the plurality of separate chambers, the at least one exchange path having a constriction disposed therein functionally separating said adjacent chambers;
at least one high voltage electrode disposed in each chamber of the plurality of separate chambers and adjacent to the constriction of the at least one exchange path;
at least one counter electrode disposed opposite the at least one high voltage electrode and adjacent to the constriction of the at least one exchange path; and
an inlet.

2. The device of claim 1, wherein the inlet is disposed at an end of the at least one exchange path, the inlet having an injection inlet, a dead volume area, an electrically active area, and at least one inlet electrode pair consisting of a high voltage electrode and a counter electrode disposed therein.

3. (canceled)

4. The device of claim 1, wherein a first constriction of the at least one exchange path is formed along a Y-axis of the cartridge, the first constriction having a first edge and a second edge, the first edge defining a chamber width, the second edge defining a channel of the at least one exchange path.

5-9. (canceled)

10. The device of claim 1, wherein the constriction of the at least one exchange path is formed along a Z-axis of the cartridge, the constriction having a rounded protrusion extending into the at least one exchange path and defining a channel of the at least one exchange path, the rounded protrusion having a radius of at least 0.5 millimeters.

11-13. (canceled)

14. The device of claim 1, wherein the at least one high voltage electrode and the at least one counter electrode are of alternating polarities.

15. The device of claim 1, further including a plurality of high voltage electrodes and a plurality of counter electrodes, wherein the plurality of high voltage electrodes and the plurality of counter electrodes are alternately arranged within the cartridge within each chamber of the plurality of separate chambers.

16-20. (canceled)

21. A method of electroporating at least one cell in a plurality of cells, comprising:
injecting a media containing the at least one cell through an inlet of a cartridge, wherein the cartridge is divided into a plurality of separate chambers;
flowing the media through the plurality of separate chambers of the cartridge, including flowing the media through at least one exchange path fluidly connecting adjacent chambers of the plurality of separate chambers;
constricting the media as it flows through the at least one exchange path via a constriction disposed within the at least one exchange path, the constriction functionally separating the adjacent chambers;
applying an electrical field to the at least one cell via at least one high voltage electrode disposed in each chamber of the plurality of separate chambers and adjacent to the constriction of the at least one exchange path, and via at least one counter electrode disposed adjacent to the constriction of the at least one exchange path and opposite the at least one high voltage electrode; and
electroporating the at least one cell.

22. The method of claim 21, further including applying a polarly opposite electrical field to the at least one cell via the at least one counter electrode.

23-25. (canceled)

26. The method of claim 21, wherein the constriction of the at least one exchange path is formed along a Z-axis of the cartridge, the constriction further having a rounded protrusion defining a channel of the at least one exchange path for constricting the media as it flows through the at least one exchange path, the rounded protrusion having a radius of at least 0.5 millimeters.

27. (canceled)

28. The method of claim 21, wherein the inlet is disposed at an end of the at least one exchange path, and the inlet further includes an injection inlet having an insertion guide, a dead volume area, and an electrically active area, and the injecting a media containing the at least one cell through the inlet of a cartridge further includes:
inserting a pipette tip into the insertion guide of the injection inlet to facilitate proper delivery of the pipette tip into the inlet; and
receiving the end of the pipette tip via an end stop disposed between the injection inlet and the dead volume area.

29. (canceled)

30. (canceled)

31. A method of transfecting at least one cell in a plurality of cells, comprising:
injecting a media containing the at least one cell and at least one nucleic acid through an inlet of a cartridge, wherein the cartridge is divided into a plurality of separate chambers;
flowing the media through the plurality of separate chambers of the cartridge, including flowing the media through at least one exchange path fluidly connecting adjacent chambers of the plurality of separate chambers;
constricting the media as it flows through the at least one exchange path via a constriction disposed within the at least one exchange path, the constriction functionally separating the adjacent chambers;
applying an electrical field to the at least one cell via at least one high voltage electrode disposed in each chamber of the plurality of separate chambers and adjacent to the constriction of the at least one exchange path, and at least one counter electrode disposed adjacent to the constriction of the at least one exchange path and opposite the at least one high voltage electrode; and
transfecting the at least one nucleic acid into the at least one cell.

32. The method of claim 31, further including applying a polarly opposite electrical field to the at least one cell via the at least one counter electrode.

33-35. (canceled)

36. The method of claim 31, wherein the constriction of the at least one exchange path is formed along a Z-axis of the cartridge, the constriction further having a rounded protrusion defining a channel of the at least one exchange path for constricting the media as it flows through the at least one exchange path, the rounded protrusion having a radius of at least 0.5 millimeters.

37. (canceled)

38. The method of claim 31, wherein the inlet is disposed at an end of the at least one exchange path, and the inlet further includes an injection inlet having an insertion guide, a dead volume area, and an electrically active area, and the injecting a media containing the at least one cell and at least one nucleic acid through the inlet of a cartridge further includes:
inserting a pipette tip into the insertion guide of the injection inlet to facilitate proper delivery of the pipette tip into the inlet; and
receiving the end of the pipette tip via an end stop disposed between the injection inlet and the dead volume area.

39. (canceled)

40. (canceled)

41. A system for electroporating at least one cell in a plurality of cells, comprising:
a cartridge divided into a plurality of separate chambers, wherein each chamber of the plurality of separate chambers is configured to hold at least one cell and is fluidly connected via at least one exchange path, the at least one exchange path having a constriction disposed therein;
at least one high voltage electrode disposed in said each chamber of the plurality of separate chambers and adjacent to the constriction of the at least one exchange path;
at least one counter electrode disposed opposite the at least one high voltage electrode and adjacent to the constriction of the at least one exchange path;
an inlet disposed at an end of the at least one exchange path, the inlet having an injection inlet, a dead volume area, an electrically active area, and at least one inlet electrode pair consisting of a high voltage electrode and a counter electrode disposed therein; and
an electroporation device configured to receive the cartridge and generate an electric pulse via the high voltage electrode and the counter electrode.

42. The system of claim 41, wherein the injection inlet further includes an insertion guide for receiving a dispenser into the inlet.

43. The system of claim 41, wherein a first constriction of the at least one exchange path is formed along a Y-axis of the cartridge, the first constriction having a first edge and a second edge, the first edge defining a chamber width, the second edge defining a channel of the at least one exchange path.

44-48. (canceled)

49. The system of claim 41, wherein the constriction of the at least one exchange path is formed along a Z-axis of the cartridge, the constriction having a rounded protrusion extending into the at least one exchange path and defining a channel of the at least one exchange path, the rounded protrusion having a radius of at least 0.5 millimeters.

50-52. (canceled)

53. The system of claim 41, wherein the at least one high voltage electrode and the at least one counter electrode are of alternating polarities.

54. The system of claim 41, further including a plurality of high voltage electrodes and a plurality of counter electrodes, wherein the plurality of high voltage electrodes and the plurality of counter electrodes are alternately arranged within the cartridge within each chamber of the plurality of separate chambers.

55-59. (canceled)
